(19) United States
(12) Patent Application Publication
(10) Pub. No.: US 2021/0186755 A1
(43) Pub. Date: Jun. 24, 2021

(54) PARALIMBAL LASER PROBE

(71) Applicant: OCUVIX PTE. LTD, Singapore (SG)

(72) Inventors: Cherry Chooi Ling KHOO, Singapore (SG); Joel M. ADUAN, Marikina City (PH)

(21) Appl. No.: 17/269,084

(22) PCT Filed: Aug. 23, 2018
(86) PCT No.: PCT/SG2018/050426

§ 371 Date: Feb. 17, 2021

(51) Int. Cl. A61F 9/009; A61F 9/008; A61F 9/007

(52) U.S. Cl. CPC A61F 9/009; A61F 9/008; A61F 9/0079; A61F 2009/00874; A61F 2009/00876; A61F 2009/00891; A61F 2009/00872; A61F 2009/00865; A61F 2009/00868

(57) ABSTRACT

A probe as disclosed herein is capable of treating both the Schlemm's canal (SC) and the trabecular meshwork (TM), or both the pars plicata and the iris root site of the eye, with electromagnetic radiation (e.g., laser light) to improve aqueous humor outflow and thus decrease intraocular pressure (IOP). The laser probe can include a tip capable of being placed on an eye, such as on a corneal limbus or a scleral limbal area of the eye. The tip can include an optical waveguide angled to direct laser light through both the SC and TM, or both the pars plicata and the iris root site of the eye. The laser light can be continuous or pulsed, and can be configured to provide appropriate therapy to both the SC and TM, or to both the pars plicata and the iris root site. The laser probe can facilitate performing trans-scleral trabeculoplasty treatment, especially trans-scleral Schlemm trabeculoplasty, and performing iridoplasty treatment.

### TECHNICAL FIELD

The present disclosure relates to ophthalmological treatments generally and more specifically to laser probes for trabeculoplasty.

### BACKGROUND

Higher-than-normal levels of intraocular pressure (TOP) is considered to be a significant factor in the development or worsening of various ocular conditions, such as glaucoma. Higher-than-normal IOP can be mitigated or avoided by increasing the aqueous humor outflow from the eye. The aqueous humor outflow path carries aqueous humor from the anterior chamber of the eye, through the trabecular meshwork (TM), and into the Schlemm's canal (SC) before flowing into the blood system.

Argon laser trabeculoplasty (ALT) and selective laser trabeculoplasty (SLT) are two techniques for treating various ocular conditions by increasing the acqueous humor outflow from the eye. Generally, ALT and SLT operate by focusing electromagnetic energy (e.g., laser light) onto the TM, which results in changes to the tissue (e.g., mechanical, chemical, biological, or otherwise) that result in increased outflow of aqueous humor, with subsequent reduction in IOP. ALT involves the use of an argon laser capable of heating the tissue upon which it is focused. SLT involves the use of a Q-switched frequency-doubled Nd:YAG laser that selectively targets and heats only the melanin granules in pigmented cells of the TM.

In operation, ALT and SLT systems generally involve focusing the laser light through a goniolens, with a skilled doctor using a slit lamp to align the laser targets at each shot location. The goniolens focuses the laser light onto TM through the anterior chamber, in a planar uveal manner. One or multiple pulses of laser light are emitted to each of these shot locations, which often include numerous spots along the TM, often covering 180° or a full 360° of the TM tissue surrounding the iris. However, shooting the laser at each of these spots along the TM can cause numerous cases of cell death along these tissues. Further, operator error can result in various complications.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a paralimbal probe, comprising: a probe tip shaped to mate with a surface of an eye at or near a corneal limbus of the eye, the eye having a Schlemm's canal and trabecular meshwork; and a waveguide positioned within the probe tip to convey electromagnetic radiation from a source of electromagnetic radiation into the eye, wherein the waveguide is oriented to direct the electromagnetic radiation along a treatment path intersecting the Schlemm's canal and the trabecular meshwork.

In some cases, the waveguide is further positioned within the probe tip such that the treatment path further intersects paralimbal tissue of the eye. In some cases, the waveguide is further positioned within the probe tip such that the treatment path further intersects corneal tissue of the eye. In some cases, the waveguide is further positioned within the probe tip such that the treatment path further intersects scleral tissue of the eye. In some cases, the waveguide is an optical waveguide and the source of electromagnetic radiation is a light source. In some cases, the source of electromagnetic radiation is a laser. In some cases, the probe tip includes a distal end shaped to mate with a curvature of the eye. In some cases, the distal end of the probe tip includes a corneal portion having a curvature that mates with a curvature of a cornea of the eye. In some cases, the distal end of the probe tip includes a scleral portion having a curvature that mates with a curvature of a sclera of the eye. In some cases, the probe further comprises one or more actuators coupled to the waveguide for adjusting the orientation of the treatment path with respect to the probe tip. In some cases, the source of electromagnetic radiation is housed within a probe body coupled to the probe tip. In some cases, the probe tip is shaped to mate with a second surface of the eye located anteriorly form the surface of the eye, and wherein the waveguide is oriented within the probe tip to direct additional electromagnetic radiation along an additional treatment path intersecting a pars plicata and an iris root site of the eye.

Embodiments of the present disclosure include an assembly, comprising: a source of electromagnetic radiation; a waveguide coupled to the source of electromagnetic radiation for conveying the electromagnetic radiation from a proximal end of the waveguide to a distal end of the waveguide; and a probe having a probe body supporting a portion of the waveguide and a probe tip supporting the distal end of the waveguide, wherein the probe tip is shaped to mate with a surface of an eye at or near a corneal limbus of the eye, and wherein the distal end of the waveguide is oriented within the probe tip to direct the electromagnetic radiation along a treatment path intersecting a Schlemm's canal and trabecular meshwork of the eye.

In some cases, the waveguide is positioned within the probe tip such that the treatment path further intersects paralimbal tissue of the eye. In some cases, the waveguide is positioned within the probe tip such that the treatment path further intersects corneal tissue of the eye. In some cases, the waveguide is positioned within the probe tip such that the treatment path further intersects scleral tissue of the eye. In some cases, the waveguide is an optical waveguide and the source of electromagnetic radiation is a light source. In some cases, the source of electromagnetic radiation is a laser. In some cases, the probe tip includes a distal end shaped to mate with a curvature of the eye. In some cases, the distal end of the probe tip includes a corneal portion having a curvature that mates with a curvature of a cornea of the eye. In some cases, the distal end of the probe tip includes a scleral portion having a curvature that mates with a curvature of a sclera of the eye. In some cases, the probe further comprises one or more actuators coupled to the waveguide for adjusting the orientation of the treatment path with respect to the probe tip.

Embodiments of the present disclosure include a scleral limbal probe, comprising: a probe tip shaped to mate with a surface of an eye at or near a scleral limbal area of the eye, the eye having a pars plicata and an iris root site; and a waveguide positioned within the probe tip to convey electromagnetic radiation from a source of electromagnetic radiation into the eye, wherein the waveguide is oriented to direct the electromagnetic radiation along a treatment path intersecting the pars plicata and the iris root site.

In some cases, the waveguide is an optical waveguide and the source of electromagnetic radiation is a light source. In some cases, the source of electromagnetic radiation is a laser. In some cases, the probe tip includes a distal end shaped to mate with a curvature of the eye. In some cases, the distal end of the probe tip includes a corneal portion having a curvature that mates with a curvature of a cornea of the eye. In some cases, the distal end of the probe tip includes a scleral portion having a curvature that mates with a curvature of a sclera of the eye. In some cases, the probe further comprises one or more actuators coupled to the waveguide for adjusting the orientation of the treatment path with respect to the probe tip. In some cases, the source of electromagnetic radiation is housed within a probe body coupled to the probe tip.

### BRIEF DESCRIPTION OF THE DRAWINGS

The specification makes reference to the following appended figures, in which use of like reference numerals in different figures is intended to illustrate like or analogous components.

FIG. 1 is a schematic diagram depicting a trans-scleral laser probe system according to certain aspects of the present disclosure.

FIG. 2 is a cut-away schematic diagram depicting a paralimbal treatment path on an eye according to certain aspects of the present disclosure.

FIG. 3 is a schematic diagram depicting a laser probe according to certain aspects of the present disclosure.

FIG. 4 is a partial cut-away schematic diagram depicting a laser probe in position on an eye according to certain aspects of the present disclosure.

FIG. 5 is a close up, partial cut-away schematic diagram depicting a laser probe treating an eye according to certain aspects of the present disclosure.

FIG. 6 is close up, cut-away schematic diagram depicting a paralimbal treatment path on an eye according to certain aspects of the present disclosure.

FIG. 7 is a schematic diagram depicting a laser probe treating the Schlemm's canal and trabecular meshwork of an eye according to certain aspects of the present disclosure.

FIG. 8 is a close up side view of a distal end of a waveguide according to certain aspects of the present disclosure.

FIG. 9 is a bottom view of a circular probe tip according to certain aspects of the present disclosure.

FIG. 10 is a bottom view of an annular sector probe tip according to certain aspects of the present disclosure.

FIG. 11 is a projection view depicting a probe tip with a waveguide in a first position according to certain aspects of the present disclosure.

FIG. 12 is a projection view depicting a probe tip with a waveguide in a second position according to certain aspects of the present disclosure.

FIG. 13 is a projection view depicting a probe tip with a waveguide in a third position according to certain aspects of the present disclosure.

FIG. 14 is a close up, partial cut-away schematic diagram depicting a laser probe performing iridoplasty on an eye according to certain aspects of the present disclosure.

### DETAILED DESCRIPTION

Certain aspects and features of the present disclosure relate to a laser probe capable of treating both the Schlemm's canal (SC) and the trabecular meshwork (TM) of the eye with electromagnetic radiation (e.g., laser light) to improve aqueous humor outflow and thus decrease intraocular pressure (IOP). The laser probe can include a tip capable of being placed on an eye, such as on a corneal limbus of the eye. The tip can include an optical waveguide angled to direct laser light through both the Schlemm's canal and trabecular meshwork of the eye. The laser light can be continuous or pulsed, and can be configured to provide appropriate therapy to both the Schlemm's canal and trabecular network. The laser probe can facilitate performing trans-scleral trabeculoplasty treatment, especially trans-scleral Schlemm trabeculoplasty.

Certain aspects and features of the present disclosure relate to a probe capable of outputting electromagnetic radiation along an output path (e.g., in an output direction). The probe can include an internal source of electromagnetic radiation (e.g., light source) or can be coupled to an external source of electromagnetic radiation (e.g., an external control box containing its own light source), such as using an optical cable.

The probe can include a probe tip. In some cases, the probe tip can be removable and can be sanitizable and/or disposable. In some cases, the entire probe can be sanitizable and/or disposable. The probe tip can be shaped to rest against an eye. In some cases, the probe tip can rest against a distinctly shaped and/or distinctly identifiable portion of the eye, such as the corneal limbus. In some cases, the probe tip can include indicators or other features to facilitate proper placement on the eye. The probe tip can be made of any suitable material for continued contact with an eye.

A waveguide (e.g., optical waveguide) can direct the electromagnetic radiation (e.g., laser light) through the probe tip and out of a distal end of the probe tip at an output angle. The output angle can be an angle other than perpendicular to the surface of the distal end of the probe tip. In some cases, the output angle can be at or approximately more than 5°, 10°, 15°, 20°, 25°, 30°, 35°, 40°, 45°, 50°, 55°, 60°, 65°, 70°, 75°, and/or 80° away from a line perpendicular to the distal end of the probe tip. Described another way, the electromagnetic radiation can exit the probe tip at an acute angle with the surface of the distal end of the probe tip.

In some cases, the waveguide or at least a portion of the waveguide can be part of a sterilizable and/or replaceable probe tip. In such cases, the waveguide or the portion of the waveguide can be insertable into a waveguide receptacle of the probe. For example, placing a sterilized or new probe tip on the probe can include inserting the waveguide or portion of the waveguide into the waveguide receptacle. The waveguide receptacle can form a continuous electromagnetic (e.g., optical) path from within the probe to the waveguide or portion of the waveguide. In some cases, however, the waveguide can be non-removable and the probe tip can be removable for sterilization and/or replacement. In some cases, the waveguide can be removable from the remainder of the probe separate from the probe tip's removability from the remainder of the probe.

While any suitable type of electromagnetic radiation can be used, the probe will be described herein as a laser probe used to deliver laser light. Therefore, as applicable, any descriptions herein attributable to laser light or optical elements can be replaced with other electromagnetic radiation and other relative elements. In some cases, the laser probe can include lenses, mirrors, or other optical elements as necessary to achieve a desired output path.

The laser probe as described herein can be used for various purposes. In at least some cases, the laser probe described herein can be especially suitable for trans-scleral trabeculoplasty treatment, such as trans-scleral Schlemm trabeculoplasty. In trans-scleral trabeculoplasty, laser light passes through the sclera of the eye to reach the trabecular meshwork. In trans-scleral Schlemm trabeculoplasty, laser light passes through the sclera of the eye along an axis that intercepts both the trabecular meshwork and the Schlemm's canal of the eye. In some cases, the probe tip as disclosed herein can have an optical waveguide oriented with respect to the distal end of the probe tip to achieve an output path of laser light that intercepts both the trabecular meshwork and the Schlemm's canal when the distal end of the probe tip is positioned against the sclera of the eye. In some cases, the probe tip as disclosed herein can have an optical waveguide oriented with respect to the distal end of the probe tip to achieve an output path of laser light that intercepts both the trabecular meshwork and the Schlemm's canal when the distal end of the probe tip is positioned against the limbus of the eye.

As used herein, the laser probe can be referred to as a paralimbal probe. The term paralimbal can refer to at or near the limbus (e.g., corneal limbus) of the eye. The treatment paths of a paralimbal probe can pass through tissue (e.g., scleral tissue and/or corneal tissue) at or adjacent the limbus before reaching the Schlemm's canal and/or trabecular meshwork.

In some cases, the laser probe is capable of providing laser light to the trabecular meshwork and/or Schlemm's canal without first sending laser light through the cornea. In some cases, the laser probe is capable of providing laser light to the trabecular meshwork and/or Schlemm's canal without sending laser light through the cornea at all. In some cases, the laser probe is capable of providing laser light to both the trabecular meshwork and the Schlemm's canal at the same time. In some cases, the laser probe is capable of providing laser light to both the trabecular meshwork and the Schlemm's canal sequentially without repositioning the laser probe. In some cases, the laser probe is capable of directing laser light along a treatment path that intersects the sclera, the Schlemm's canal, and then the trabecular meshwork.

In some cases, actuators in the probe can induce movement of the output path of the laser light. In some cases, actuators can manipulate the waveguide to change the output path of the laser light. In some cases, actuators can manipulate other elements of the probe to adjust the output path of the laser light. The output path of the laser light can be thus adjusted without needing to move and/or reposition the probe tip on the eye. Thus, different locations of the eye can be treated without needing to move and/or reposition the probe tip on the eye.

Certain aspects and features of the present disclosure may be especially suitable for treating not only the Schlemm's canal and the trabecular meshwork, but also simultaneously treating more of the trabecular outflow pathway than possible with ALT or SLT systems. For example, a laser directed through the Schlemm's canal and the trabecular meshwork along a paralimbal treatment path, such as described herein, can also impinge upon the intertrabecular tissue and permit treatment of the whole trabecular apparatus, which can lead to improved (e.g., increased) aqueous outflow.

Certain aspects and features of the present disclosure may be especially suitable for iridoplasty, such as to treat plateau iris syndrome or angle closure glaucoma (ACG). Certain aspects and features of the present disclosure can enable a treatment path that directs laser light through the limbal scleral area towards the pars plicata and iris root site to cause an iridoplasty effect as well as a therapeutic laser contracture to the anteriorly positioned ciliary process or pars plicata area, thereby causing a posterior movement of the ciliary process away from the iris root and opening the trabecular angle. For example, the same laser probe used for treatment of the Schlemm's canal and trabecular meshwork can be shifted posteriorly by approximately 1-3 mm to redirect the laser light to the anterior portion of the ciliary body (e.g., pars plicata) and iris root. The laser light can induce mild contracture burns to these ocular structures to widen the anterior chamber angles. In some cases, a laser power of approximately 1-1.6 Watts with a duty cycle of approximately 30-45% and a duration of approximately 50-80 seconds can be used, although other settings can be used.

In some cases, certain aspects of the present disclosure enable treatment of plateau iris syndrome caused by anteriorly positioned ciliary process, for which other iridoplasty techniques are incapable of treating.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

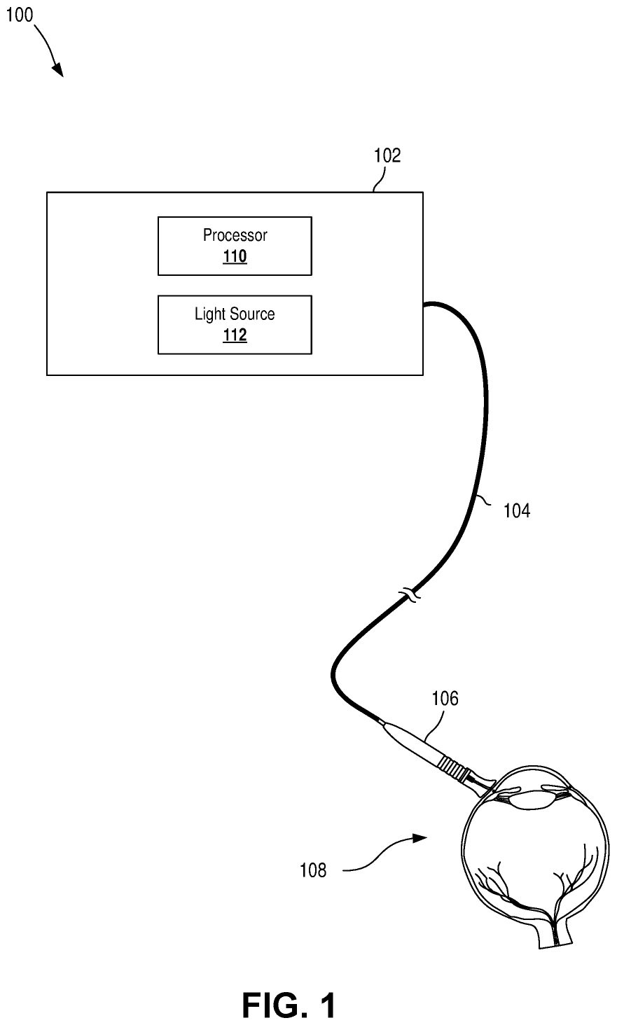

FIG. 1 is a schematic diagram depicting a trans-scleral laser probe system 100 according to certain aspects of the present disclosure. The laser probe system 100 can include a control box 102 coupled to a probe 106 via a probe cable 104. The control box 102 can include a processor 110 and light source 112, as well as other suitable equipment not shown for illustrative purposes (e.g., power supply, memory, interfaces, and the like). The light source 112 can be a laser light source. The probe cable 104 can be an optical cable capable of conveying the light from the light source 112 to the probe 106. In some cases, the probe cable 104 can include electrical connections to convey power and/or data signals between the control box 102 and the probe 106. In some cases, the probe 106 can contain its own light source, in which case the probe cable 104 may carry power to power the light source and may not carry any optical signals.

The probe 106 can be positioned on an eye 108 to treat the eye as described herein. The probe 106 depicted in FIG. 1 can be of any suitable shape or size, and not necessarily as depicted.

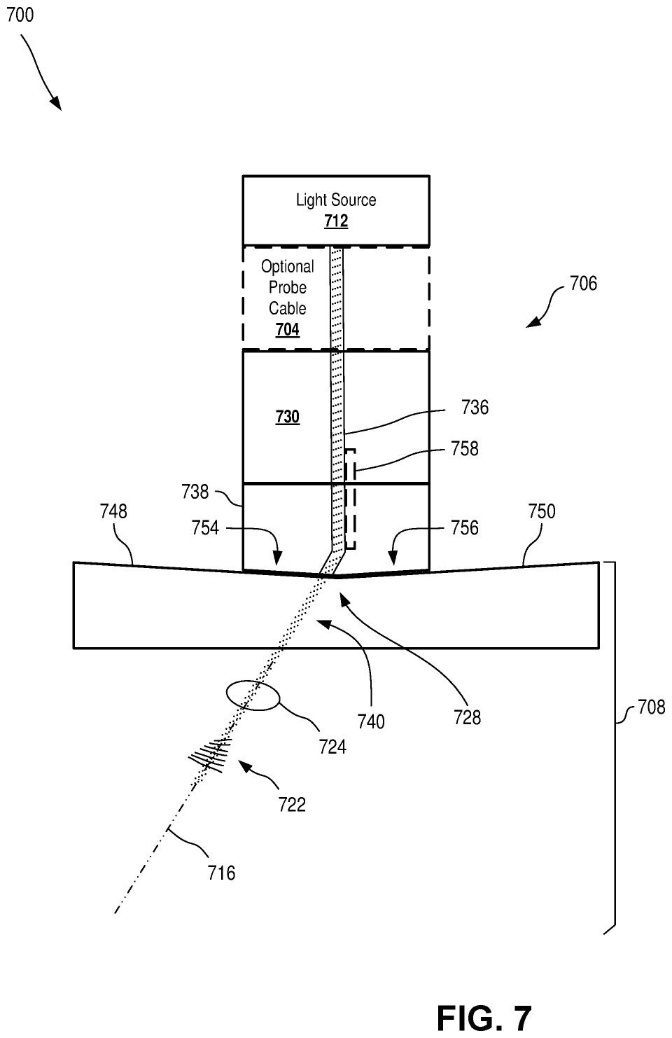

In some cases, processor 110 can enable automation of the treatment process. Automation can include automatically adjusting laser settings (e.g., power, duty cycle, frequency, duration, or others), automatically adjusting the laser treatment path during or between treatments (e.g., using actuators or paths with reference to FIGS. 7 and 11-13), and/or automatically triggering laser light output, such as in response to sensor input indicating desired positioning of the probe 106.

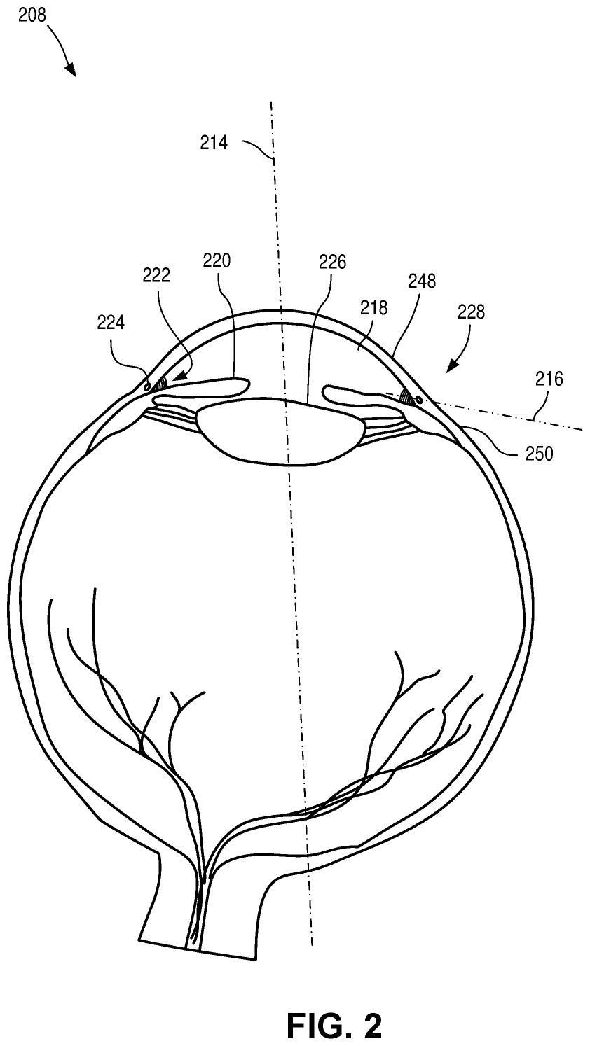

FIG. 2 is a cut-away schematic diagram depicting a paralimbal treatment path 216 on an eye 208 according to certain aspects of the present disclosure. The eye 208 can be eye 108 of FIG. 1. The eye 208 can include an anterior chamber 218 containing aqueous humor. The eye includes an iris 220 and lens 226. The corneal limbus 228 can be located at the border between the cornea 248 and sclera 250. Near the base of the cornea 248, the trabecular meshwork 222 can be found between cornea 248 and the iris 220 to facilitate drainage of aqueous humor from the anterior chamber 218 into the Schlemm's canal 224. The approximate optical axis 214 of the eye 208 is shown.

According to certain aspects of the present disclosure, laser treatment can be provided through the sclera 250 along a treatment path 216 that intersects both the Schlemm's canal 224 and the trabecular meshwork 222. Due to the size and position of the Schlemm's canal 224 and trabecular meshwork 222, it can be difficult or impossible to treat both locations simultaneously using a goniolens, which would rest on the cornea 248 and be generally centered on the optical axis 214. However, a laser probe positioned at the limbus 228 can direct laser light through both the Schlemm's canal 224 and trabecular meshwork 222, such as along treatment path 216. It will be understood that various treatment paths may exist other than the treatment path 216 depicted in FIG. 2, as long as the treatment paths intersect both the Schlemm's canal 224 and trabecular meshwork 222.

Generally, as disclosed herein, treatment can be provided from a laser probe positioned at the limbus 228 by transmitting laser light through the sclera 216, through the Schlemm's canal 224, then to the trabecular meshwork 222, all at portions located adjacent the laser probe (e.g., without the laser light passing the optical axis 214 prior to contacting the Schlemm's canal 224 and trabecular meshwork 222). However, in some cases, the laser probe can be constructed to provide laser light to portions of the trabecular meshwork 222 and Schlemm's canal 224 located opposite optical axis 214 from where the laser probe is positioned. In such cases, the treatment path may extend first through the cornea 248 adjacent the limbus 228, through the optical axis 214 of the eye 208, then through a portion of the trabecular meshwork 222 at the opposite side of the anterior chamber 218, then to the Schlemm's canal 224 adjacent that portion of trabecular meshwork 222.

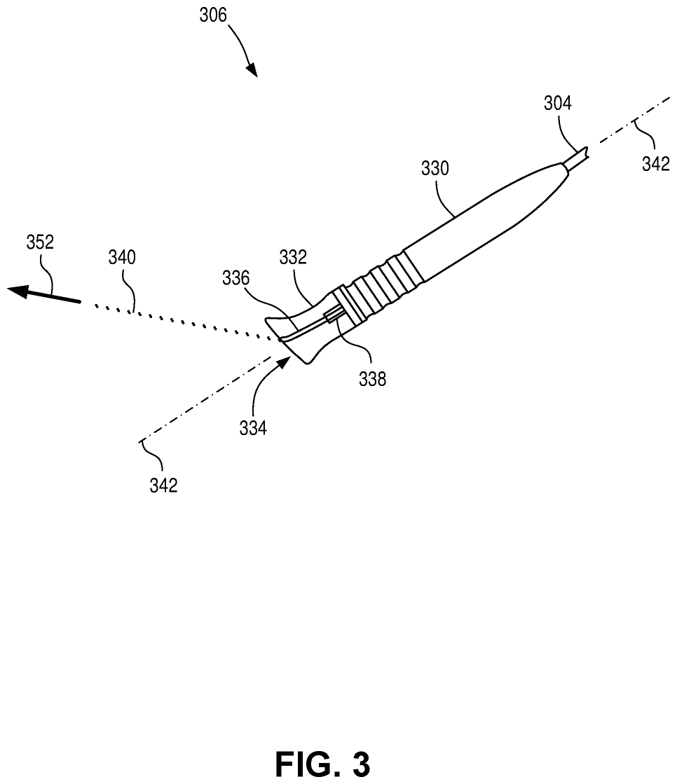

FIG. 3 is a schematic diagram depicting a laser probe 306 according to certain aspects of the present disclosure. The laser probe 306 can be the laser probe 106 of FIG. 1. The laser probe 306 can be any suitable shape or size any may not necessarily appear as depicted in FIG. 3. The laser probe 306 can include a probe body 330 and a probe tip 332. Treatment radiation, such as laser light 340, can exit the probe 306 via waveguide 336. Waveguide 336 can be an optical waveguide for transmitting the laser light 340. The laser light 340 can be generated by a light source that is internal to the probe 306 (e.g., housed within the probe body 330) or external to the probe 306 (e.g., housed in a control box and carried to the probe 306 via probe cable 304). Probe cable 304 can convey energy to the probe 306, such as optical energy (e.g., in the case of the light source being housed in an external control box) or electrical energy (e.g., to power an internal light source of the probe 306).

The probe tip 332 can include a distal end 334. The distal end 334 can be designed to be placed against an eye, such as against the limbus of an eye (e.g., limbus 228 of eye 208 of FIG. 2). The distal end 334 of the probe tip 332 can be shaped to be placed against a surface. The distal end 334 of the probe tip 332 can have a diameter sized to facilitate easy placement of the probe tip 332 against the limbus of an eye, which can include a distal end 334 small enough to not act as an obstruction during placement and maneuvering against the limbus of the eye, while also being large enough to provide sufficient surface area to maintain steady and reliable contact with the eye during treatment. In some cases, the distal end 334 of the probe tip 332 can have a diameter of approximately 2 mm to 5 mm, approximately 3 mm to 4 mm, or approximately 3.5 mm. In some cases, however, the distal end 334 can have a diameter that is smaller than 2 mm or larger than 5 mm.

The probe tip 332 can have a length suitable to distance the probe body 330 from the eye during treatment to avoid contamination and contact between the probe body 330 and the patient (e.g., the eye, eyelids, mucus membranes, or other parts of the patient). In some cases, the probe tip 332 can advantageously have a length of between approximately 3 mm and 7 mm, between approximately 4 mm and 6 mm, or approximately 5 mm. In some cases, the probe tip 332 can have a length of at or at least approximately 3 mm, 4 mm, 5 mm, 6 mm, or 7 mm.

The entire laser probe 306, from distal end 334 of the probe tip 332 to the proximal end of the probe body 330 can have any suitable length, such as to facilitate dexterous, manual handling by a treatment professional. In some cases, this length can be between approximately 70 mm and 90 mm, between approximately 75 mm and 85 mm, or approximately 80 mm. In some cases, however, this length can be less than 70 mm or greater than 90 mm. The probe body 330 can also have any suitable diameter, such as to facilitate dexterous, manual handling by a treatment professional. In some cases, this diameter can be between approximately 10 mm and 20 mm, approximately 12 mm and 18 mm, or approximately 15 mm. In some cases, however, the diameter can be smaller than 10 mm or greater than 20 mm.

A probe axis 342 can be an axis that is normal or substantially normal (e.g., within 0.5°, 1°, 1.5°, 2°, 2.5°, 3°, 3.5°, 4°, 4.5°, 5°, 6°, 7°, 8°, 9°, or 10° of normal or less) to the surface against which the distal end 334 is to be placed or to the surface of the distal end 334. In some cases, the probe axis 342 can extend axially along the probe body 330, although that need not always be the case. The probe axis 342 can also be referred to as a probe placement axis.

Laser light 340 can be transmitted through the waveguide 336 and output in a direction 352. Laser light 340 can enter the waveguide 336 at a proximal end (not shown), such as at a light source, and can be conveyed by the waveguide 336 until it exits the waveguide 336 at a distal end (e.g., at or near the distal end 334 of the probe tip 332). The waveguide 336 can be shaped and/or oriented to direct laser light 340 in direction 352 such that an angle exists between the probe axis 342 and the direction 352. The angle can be at or approximately more than 5°, 10°, 15°, 20°, 25°, 30°, 35°, 40°, 45°, 50°, 55°, 60°, 65°, 70°, 75°, and/or 80°, and less than 90°. Put another way, direction 352 can form an acute angle with the surface of the distal end 334 of the probe tip 332. In some cases, the waveguide 336 can extend generally parallel to the probe axis 342 for an extent before angling towards direction 352 before reaching the distal end 334 of the probe tip 332.

In some cases, the probe tip 332 is removable from the probe body 330, such as for sterilization and/or replacement. In some cases, the waveguide 336 can be integrated with the probe tip 332 and be removable from the probe body 330 along with the probe tip 332. In some cases, the waveguide 336 can be located within an opening of the probe tip 332 and the probe tip 332 can be removable from the probe body 330 without requiring removal of the waveguide 336 from the probe body 330. In such cases, the waveguide 336 may be removable from the probe body 330 or not. The waveguide 336 can be secured to the probe body 336 at a waveguide receptacle 338. In some cases, if the waveguide 336 is removable, the waveguide receptacle 338 can accept the waveguide 336 and establish an optical coupling to permit laser light 340 to be directed from within the probe body 330 to the waveguide 336 and out the waveguide 336.

The probe 306 can additionally contain additional elements, such as actuators, switches, cameras, sensors, or the like. In some cases, these additional elements can facilitate probe placement (e.g., appropriate placement at the limbus) or use (e.g., actuation of the control box to initiate outputting of the laser light 340). In some cases, probe 306 can include additional actuators capable of manipulating the direction 352 of the laser light 340, at least with respect to the probe axis 342. In such cases, the direction 352 of the laser light 340 can be manipulated without needing to remove the probe tip 332 from the eye or otherwise move the probe tip 332. In some cases, such additional actuators can rotate the waveguide 336 to adjust the direction 352 around the probe axis 342. In some cases, additional actuators can further adjust the angle between the direction 352 and probe axis 342, such as by manipulating the orientation of the waveguide 336 within the probe tip 332.

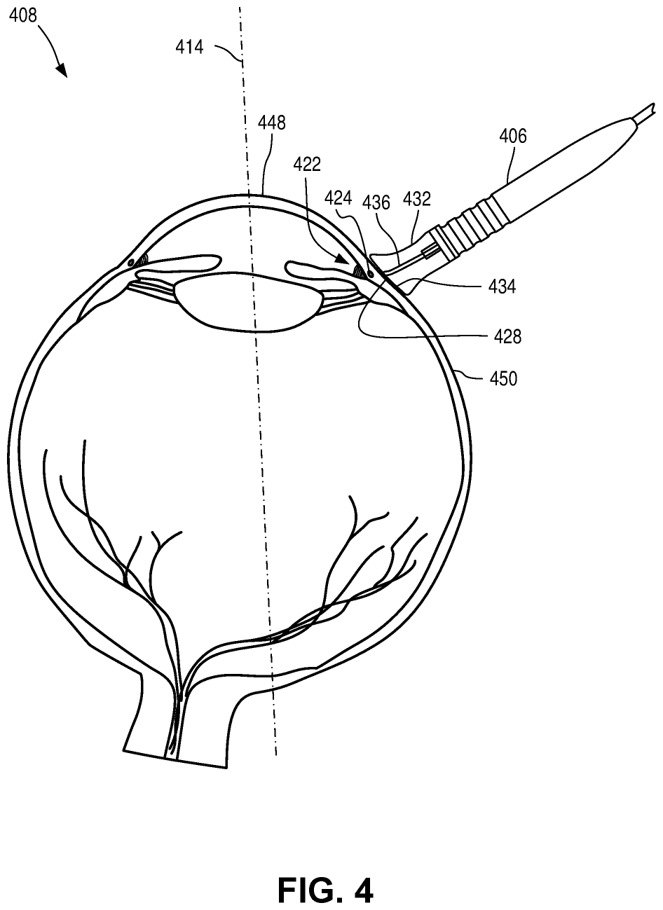

FIG. 4 is a partial cut-away schematic diagram depicting a laser probe 406 in position on an eye 408 according to certain aspects of the present disclosure. The laser probe 406 and eye 408 can be laser probe 106 and eye 108 of FIG. 1, respectively. The laser probe 406 can be placed such that the distal end 434 of the probe tip 432 rests against the limbus 428 of the eye 408. The distal end 434 of the probe tip 432 can be shaped to facilitate proper placement of the probe tip 432 at the limbus 428 of the eye 408. The waveguide 436 of the probe 406 can be oriented to deliver laser light through the Schlemm's canal 424 and the trabecular meshwork 422. In some cases, the waveguide 436 of the probe 406 can be oriented to deliver laser light through the Schlemm's canal 424 and the trabecular meshwork 422 without first passing the optical axis 414 of the eye 408. In some cases, the waveguide 436 of the probe 406 can be oriented to deliver laser light through the Schlemm's canal 424 and the trabecular meshwork 422 after first passing through paralimbal tissue of the eye 408. In some cases, the paralimbal tissue can include tissue of the cornea 448. In some cases, the paralimbal tissue can include tissue of the sclera 450.

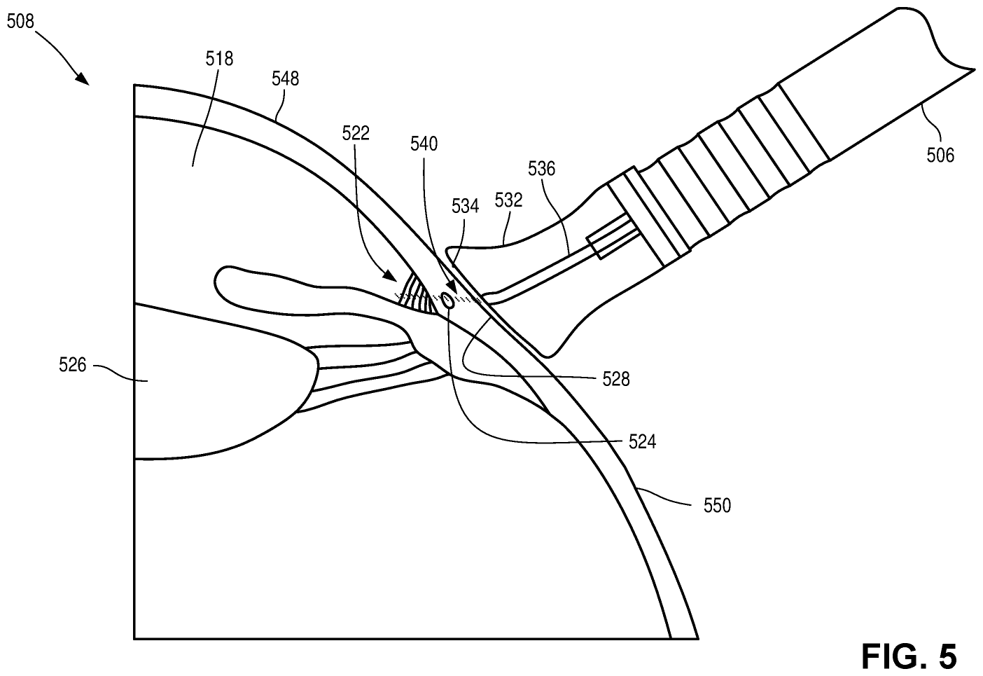

FIG. 5 is a close up, partial cut-away schematic diagram depicting a laser probe 506 treating an eye 508 according to certain aspects of the present disclosure. The laser probe 506 and eye 508 can be laser probe 106 and eye 108 of FIG. 1, respectively. The lens 526 and anterior chamber 518 are identified for illustrative purposes.

The laser probe 506 can be placed such that the distal end 534 of the probe tip 532 rests against the limbus 528 of the eye 508. The distal end 534 of the probe tip 532 can be shaped to facilitate proper placement of the probe tip 532 at the limbus 528 of the eye 508. The waveguide 536 of the probe 506 can be oriented to deliver laser light 540 through the Schlemm's canal 524 and the trabecular meshwork 522. In some cases, the waveguide 536 of the probe 506 can be oriented to deliver laser light through the Schlemm's canal 524 and the trabecular meshwork 522 without first passing the optical axis 514 of the eye 508. In some cases, the waveguide 536 of the probe 506 can be oriented to deliver laser light through the Schlemm's canal 524 and the trabecular meshwork 522 after first passing through paralimbal tissue of the eye 508. In some cases, the paralimbal tissue can include tissue of the cornea 548. In some cases, the paralimbal tissue can include tissue of the sclera 550.

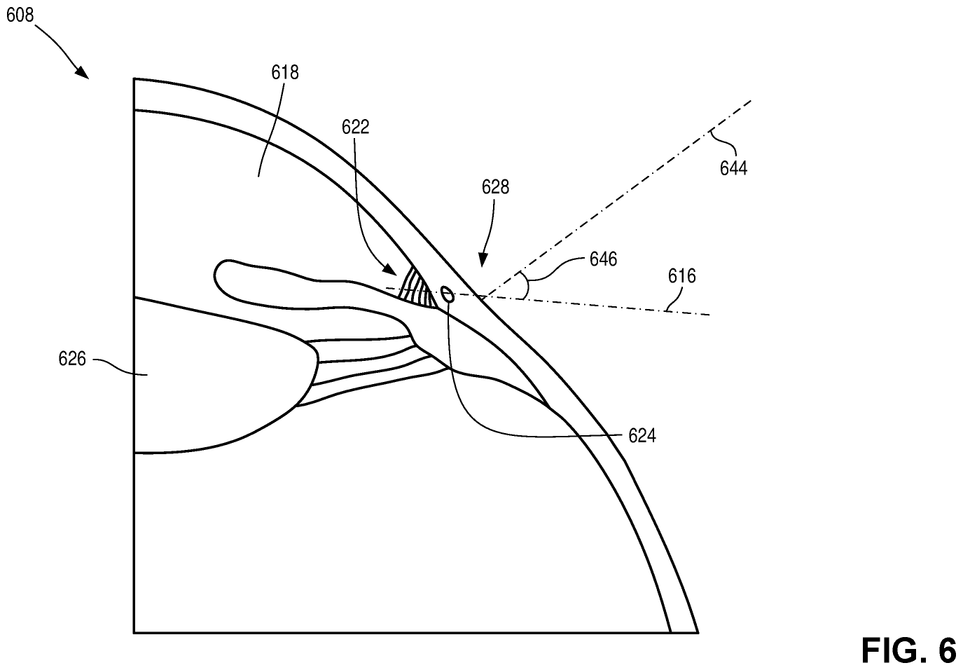

FIG. 6 is close up, cut-away schematic diagram depicting a paralimbal treatment path 616 on an eye 608 according to certain aspects of the present disclosure. The eye 608 can be eye 108 of FIG. 1. The lens 626 and anterior chamber 618 are identified for illustrative purposes. The paralimbal treatment path 616 extends through both the Schlemm's canal 624 and trabecular meshwork 622 of the eye 608. The paralimbal treatment path 616 also passes through tissue at or near the corneal limbus 628, such as corneal tissue or scleral tissue. In some cases, the paralimbal treatment path 616 passes through scleral tissue at or near the corneal limbus 628 and does not pass through corneal tissue.

Line 644 represents an axis normal or substantially normal (e.g., within 0.5°, 1°, 1.5°, 2°, 2.5°, 3°, 3.5°, 4°, 4.5°, 5°, 6°, 7°, 8°, 9°, or 10° of normal or less) to the limbus 628 of the eye 608 (e.g., the surface of the eye 608 at the limbus 628). Line 644 can be referred to as a limbal-normal axis. The paralimbal treatment path 616 can form an angle 646 with line 644. The angle 646 can be an acute angle. The angle 646 can be less than 90° and at or greater than approximately 1°, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, and/or 89°. In some cases, angle 646 can have other values.

As described herein, a laser probe (e.g., laser probe 106 of FIG. 1) can be configured to emit laser light along the paralimbal treatment path 616 when the laser probe is positioned on or near the limbus 628 of the eye 608.

FIG. 7 is a schematic diagram depicting a laser probe 706 treating the Schlemm's canal 724 and trabecular meshwork 722 of an eye according to certain aspects of the present disclosure. The laser probe 706 can include a probe body 730, a probe tip 738, and a waveguide 736. A light source 712 can generate laser light 740 that is fed into the waveguide 736 and output at the probe tip 738 along a treatment path 716 that intersects both the Schlemm's canal 724 and the trabecular meshwork 722 of an eye 708.

The probe tip 738 can be shaped to mate with the contours of the limbus 728 of the eye 708. For example, as depicted in FIG. 7, the surface of the eye 708 at the limbus 728 can have a slight bend or groove formed where the curvature of the corneal tissue 748 meets the different curvature of the scleral tissue 750. The distal end of the probe tip 738 can be shaped to mate with the surface of the eye 708 at the limbus 728, such as by including a corneal portion 754 having a curvature that mates with (e.g., matches) the curvature of the corneal tissue 748 at or near the limbus 728; as well as a scleral portion 756 having a curvature that mates with (e.g., matches) the curvature of the scleral tissue 750. Thus, the probe tip 738 can be shaped to facilitate placement of the probe tip 738 in a proper position at the limbus 728 of the eye 708.

The waveguide 736 of the laser probe 706 can be shaped to output light along the treatment path 716 when the probe tip 738 is in proper position. For example, the waveguide 736 of the laser probe 706 can direct laser light in a direction that intersects both the Schlemm's canal 724 and the trabecular meshwork 722 when the corneal portion 754 of the distal end of the probe tip 738 mates with the corneal tissue 748 at or near the limbus 728 and when the scleral portion 756 of the distal end of the probe tip 738 mates with the scleral tissue 750 at or near the limbus 728. While depicted as centered within the probe body 730 of the laser probe 706 in FIG. 7, the waveguide 736 can be positioned anywhere within or on the laser probe 706. In some cases, the waveguide is centered at the limbus 728 (e.g., centered on an axis intersecting the limbus 728) for at least a portion of the length of the laser probe 706 before being tilted towards an axis collinear with the treatment path 716.

In some cases, the light source 712 can be part of the laser probe 706. In some cases, the light source 712 can be separate from the laser probe 706 and can be coupled to the laser probe 706 via a probe cable 704.

In some cases, an optional actuator 758 can be coupled to the waveguide 736 and/or the probe tip 738 to manipulate the waveguide 736 to facilitate adjusting the treatment path 716 without needing to otherwise move the probe tip 738 with respect to the eye 708. The actuator 758 may extend into both the probe body 730 and the probe tip 738, may exist solely within the probe body 738, or may exist solely within the probe tip 738. The actuator 758 can induce any suitable motion in the waveguide 736 to direct the laser light 740 in the desired direction, such as a bending motion or a rotary motion. Any suitable type of actuator 758 can be used to impart force on the waveguide 736 to adjust the output path of the laser light 740, such as a rotary actuator (e.g., a motorized collar attached to the waveguide 736 to turn the waveguide 736), a linear actuator (e.g., a screw-type actuator to push and/or pull the waveguide 736), a non-contacting actuator (e.g., an electromagnet magnetically coupled to a corresponding magnetic structure coupled to the waveguide 736 to pull the waveguide 736 in response to an applied magnetic field), a bending actuator (e.g., a piezoelectric bending actuator to bend the waveguide 736 in response to an applied electrical signal), or any other suitable actuators. In some optional cases, the output path of the laser light 740 can be steered using other techniques, such as lenses or optical phase arrays.

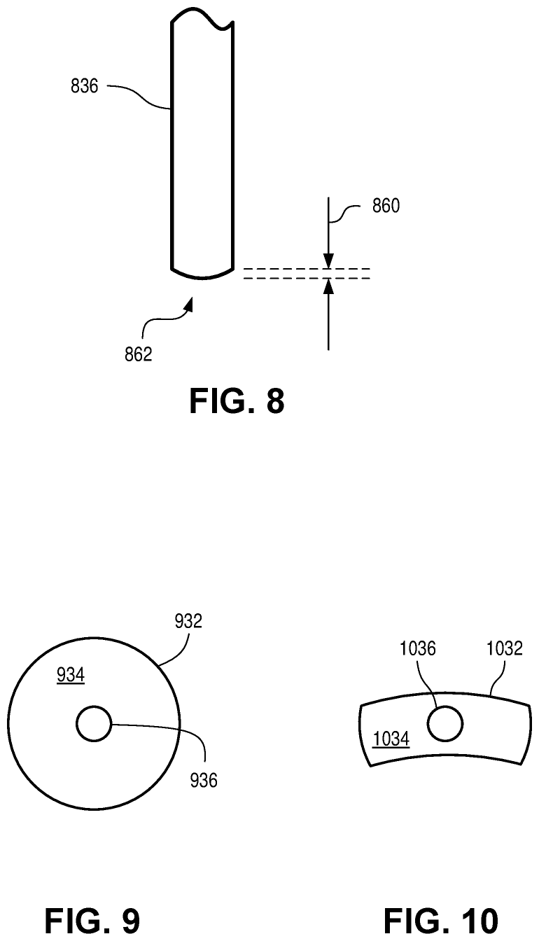

FIG. 8 is a close up side view of a distal end 862 of a waveguide 836 according to certain aspects of the present disclosure. Waveguide 836 can be waveguide 336 of FIG. 3. The waveguide 836 is shown without the surrounding probe tip and with exaggerated dimensions for illustrative purposes. The distal end 862 of the waveguide 836 can have a curvature shaped to match the limbus of an eye (e.g., limbus 228 of eye 208). In some cases, the curvature of the distal end 862 of the waveguide 836 can have a curvature height 860 that is at or approximately between 0.5 mm and 1.5 mm, between 0.75 mm and 1.25 mm, or at or approximately 1 mm, In some cases, other curvature heights 860 can be used.

FIG. 9 is a bottom view of a circular probe tip 932 according to certain aspects of the present disclosure. The probe tip 932 can have a generally circular shape or cross section at least at the distal end 934 of the probe tip 932. The waveguide 936 can exit from the center of the distal end 934 of the probe tip 932, although that need not always by the case.

FIG. 10 is a bottom view of an annular sector probe tip 1032 according to certain aspects of the present disclosure. The probe tip 1032 can have a slim, curved shape or cross section that generally takes the form of an annular sector (e.g., a sector or section of an annulus) at least at the distal end 1034 of the probe tip 1032. As depicted in FIG. 10, the sides of the annular sector can shape can be rounded or otherwise shaped to facilitate manufacturing and/or reduce risk of injury when used near the eye. In some cases, the curvature of the top and bottom edges (e.g., as oriented in FIG. 10) of the annular shape can have approximately the same curvature (e.g., as seen in FIG. 10), or can have largely different curvatures. In some cases, the curvature of the annular sector shape can match or approximate the general curvature of the limbus of an eye (e.g., limbus 228 of eye 208). The waveguide 1036 can exit from the center of the distal end 1034 of the probe tip 1032, although that need not always by the case.

In some cases, the cross section or distal end of a probe tip can have shapes other than circular or similar to an annular sector.

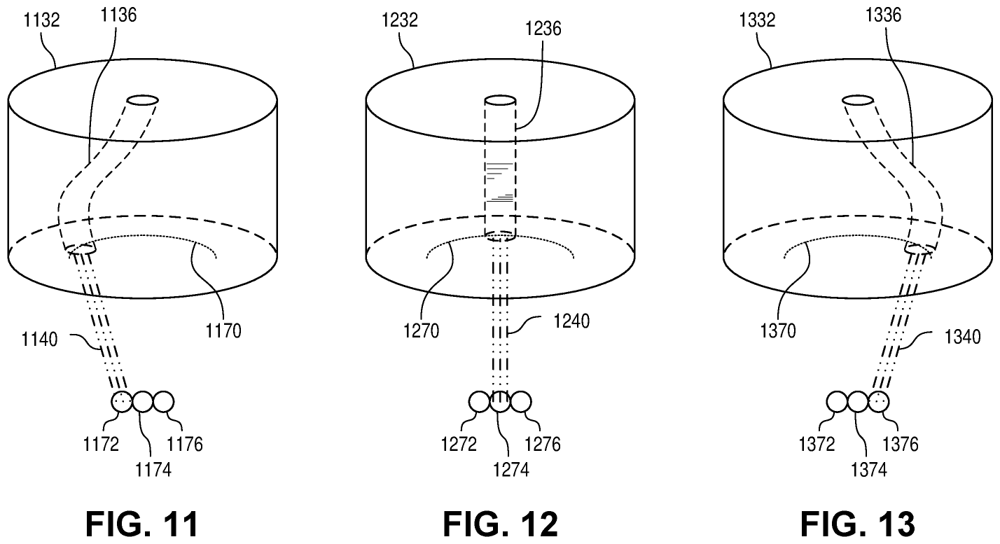

FIG. 11 is a projection view depicting a probe tip 1132 with a waveguide 1136 in a first position according to certain aspects of the present disclosure. The probe tip 1132 can be probe tip 332 of FIG. 3 or any other suitable probe tip. The probe tip 1132 can be used with any suitable probe body or can be incorporated into a probe body. The probe tip 1132 can contain a waveguide 1136 therein.

The waveguide 1136 can be movable through multiple positions without needing to move the placement of the probe tip 1132 on an eye, thus permitting multiple locations to be treated without needing to reposition the probe tip 1132 on the eye. In some cases, the probe tip 1132 can rotate or include rotatable portions to facilitate movement of the waveguide 1136 between different positions. In some cases, the probe tip 1132 can remain stationary as the waveguide 1136 is moved within the probe tip 1132. Movement can be achieved using manual mechanical controls (e.g., manipulation of rotatable parts to rotate a portion of the waveguide 1136), user-activated electronic controls (e.g., pressing of a button to cause an actuator to move the waveguide 1136), automated electronic controls (e.g., a computer program that causes an actuator to automatically move the waveguide 1136), or otherwise.

The waveguide 1136 can be manipulated in any suitable fashion, such as described herein. In some cases, the various positions of the waveguide 1136 can follow a path 1170. In some cases, features of the probe tip 1132 or other aspects of the probe can restrict movement of the waveguide 1136 to positions along the path 1170. In an example, the waveguide 1136 can be positioned in a cutout portion of the probe tip 1132 that restricts movement of waveguide 1136 to only positions along the path 1170. In another example, a rail or track can be coupled to or positioned adjacent the waveguide 1136 to restrict movement of waveguide 1136 to only positions along the path 1170. In some cases, an actuator controlling movement of the waveguide 1136 can use mechanical linkages to restrict movement of the waveguide 1136 to only positions along the path 1170. In some cases, software controls can be used with an actuator to ensure the waveguide 1136 is only moved to positions along the path 1170. Other techniques can be used to control positioning of the waveguide 1136.

As depicted in FIG. 11, the waveguide 1136 can have a bent shape that permits rotation of the waveguide 1136 around the center axis of the probe tip 1132 to move the output end of the waveguide 1136 along path 1170. In some cases, the waveguide 1136 can maintain a consistent angle of output laser light 1140 from different positions along the path 1170.

In the first position, as depicted in FIG. 11, the waveguide 1136 can be oriented in a fashion that directs laser light 1140 into a first treatment area 1172. A second treatment area 1174 and third treatment area 1176 may remain untreated by the laser light 1140 when the waveguide 1136 is in the first position, although that need not always be the case.

As used herein, a treatment area can include any suitable tissue for treatment, such as portions of a Schlemm's canal and/or portions of trabecular meshwork. In some cases, multiple treatment areas associated with different positions of the waveguide can be distinct (e.g., not overlapping). However, in some cases, multiple treatment areas associated with different positions of the waveguide can be partially overlapping. In some cases, the waveguide 1136 and path 1170 can be configured such that different positions can treat multiple treatment areas that are associated with the same portion of trabecular network but different portions of the Schlemm's canal. In other cases, the waveguide 1136 and path 1170 can be configured such that different positions can treat multiple treatment areas that are associated with different portions of trabecular network and different portions of the Schlemm's canal

After treatment of first treatment area 1172 by the laser light 1140, the laser light 1140 can be optionally stopped and the waveguide 1136 can be moved to another position, such as position two, as depicted in FIG. 12.

FIG. 12 is a projection view depicting a probe tip 1232 with a waveguide 1236 in a second position according to certain aspects of the present disclosure. The probe tip 1232 can be probe tip 1132 of FIG. 11 after being moved into a second position, or can be any other suitable probe tip. The probe tip 1232 can be used with any suitable probe body or can be incorporated into a probe body. The probe tip 1232 can contain a waveguide 1236 therein.

In the second position, as depicted in FIG. 12, the waveguide 1236 can be oriented in a fashion that directs laser light 1240 into a second treatment area 1274. A first treatment area 1272 and third treatment area 1276 may remain untreated by the laser light 1240 when the waveguide 1236 is in the second position, although that need not always be the case.

After treatment of second treatment area 1274 by the laser light 1240, the laser light 1240 can be optionally stopped and the waveguide 1236 can be moved to another position, such as position three, as depicted in FIG. 13.

FIG. 13 is a projection view depicting a probe tip 1332 with a waveguide 1336 in a third position according to certain aspects of the present disclosure. The probe tip 1332 can be probe tip 1232 of FIG. 12 after being moved into a third position, or can be any other suitable probe tip. The probe tip 1332 can be used with any suitable probe body or can be incorporated into a probe body. The probe tip 1332 can contain a waveguide 1336 therein.

In the third position, as depicted in FIG. 13, the waveguide 1336 can be oriented in a fashion that directs laser light 1340 into a third treatment area 1376. A first treatment area 1372 and second treatment area 1374 may remain untreated by the laser light 1340 when the waveguide 1336 is in the third position, although that need not always be the case.

After treatment of third treatment area 1376 by the laser light 1340, the laser light 1340 can be stopped and the probe tip 1132 can be repositioned on the eye to treat additional treatment areas.

While FIGS. 11-13 describe three positions, it will be understood that any number of positions can be used and in any desirable order or combination to treat a desired set of treatment areas. In some cases, the same position can be used more than once to provide continued treatment to the same treatment area without repositioning the probe tip. In such cases, multiple instances of treatment of the same treatment area can be separated by treatment of another treatment area to permit the first treatment area to heal or cool down between treatments, all without repositioning the probe tip.

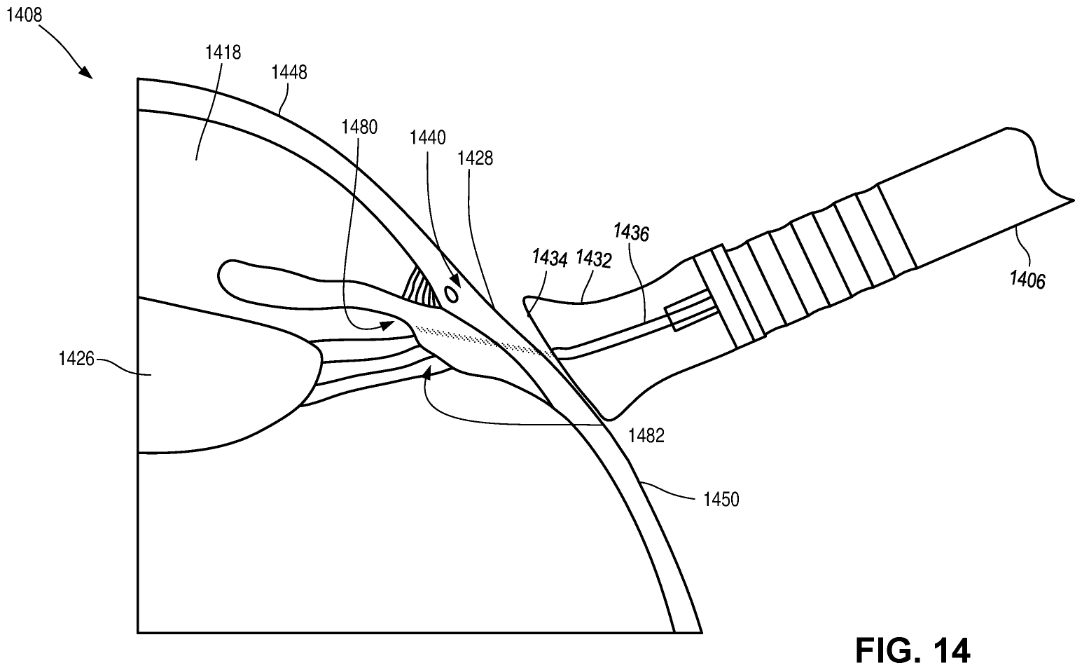

FIG. 14 is a close up, partial cut-away schematic diagram depicting a laser probe 1406 performing iridoplasty on an eye 1408 according to certain aspects of the present disclosure. In some cases, the iridoplasty depicted in FIG. 14 can be considered iridoparsplicataplasty due to the simultaneous treatment of the pars plicata and iris root. The laser probe 1406 and eye 1408 can be laser probe 106 and eye 108 of FIG. 1, respectively. The lens 1426 and anterior chamber 1418 are identified for illustrative purposes.

The laser probe 1406 can be placed such that the distal end 1434 of the probe tip 1432 rests against the scleral 1450 at or near the limbus 1428 of the eye 1408 (e.g., a scleral limbal area). The distal end 1434 of the probe tip 1432 can be shaped to facilitate proper placement of the probe tip 1432 at the scleral limbal area. In some cases, the probe tip 1432 can be shaped to facilitate placement of the probe tip 143 at both the scleral limbal area and the limbus 1428 of the eye 1408 (e.g., to facilitate both treatment of the Schlemm's canal and trabecular meshwork and treatment of the pars plicata and iris root. The waveguide 1436 of the probe 1406 can be oriented to deliver laser light 1440 to the pars plicata 1482 and the iris root 1480. In some cases, the waveguide 1436 of the probe 1406 can be oriented to deliver laser light through the pars plicata 1482 and the iris root 1480 without first passing the optical axis 1414 of the eye 1408. In some cases, the waveguide 1436 of the probe 1406 can be oriented to deliver laser light through the pars plicata 1482 and the iris root 1480 after first passing through scleral tissue of the eye 1408.

As used below, any reference to a series of examples is to be understood as a reference to each of those examples disjunctively (e.g., “Examples 1-4” is to be understood as “Examples 1, 2, 3, or 4”).

Example 1 is a paralimbal probe, comprising: a probe tip shaped to mate with a surface of an eye at or near a corneal limbus of the eye, the eye having a Schlemm's canal and trabecular meshwork; and a waveguide positioned within the probe tip to convey electromagnetic radiation from a source of electromagnetic radiation into the eye, wherein the waveguide is oriented to direct the electromagnetic radiation along a treatment path intersecting the Schlemm's canal and the trabecular meshwork.

Example 2 is the probe of example(s) 1, wherein the waveguide is further positioned within the probe tip such that the treatment path further intersects paralimbal tissue of the eye.

Example 3 is the probe of example(s) 1 or 2, wherein the waveguide is further positioned within the probe tip such that the treatment path further intersects corneal tissue of the eye.

Example 4 is the probe of example(s) 1 or 2, wherein the waveguide is further positioned within the probe tip such that the treatment path further intersects scleral tissue of the eye.

Example 5 is the probe of example(s) 1-4, wherein the waveguide is an optical waveguide and the source of electromagnetic radiation is a light source.

Example 6 is the probe of example(s) 5, wherein the source of electromagnetic radiation is a laser.

Example 7 is the probe of example(s) 1-6, wherein the probe tip includes a distal end shaped to mate with a curvature of the eye.

Example 8 is the probe of example(s) 7, wherein the distal end of the probe tip includes a corneal portion having a curvature that mates with a curvature of a cornea of the eye.

Example 9 is the probe of example(s) 7 or 8, wherein the distal end of the probe tip includes a scleral portion having a curvature that mates with a curvature of a sclera of the eye.

Example 10 is the probe of example(s) 1-9, further comprising one or more actuators coupled to the waveguide for adjusting the orientation of the treatment path with respect to the probe tip.

Example 11 is the probe of example(s) 1-10, wherein the source of electromagnetic radiation is housed within a probe body coupled to the probe tip.

Example 12 is the probe of example(s) 1-11, wherein the probe tip is shaped to mate with a second surface of the eye located anteriorly form the surface of the eye, and wherein the waveguide is oriented within the probe tip to direct additional electromagnetic radiation along an additional treatment path intersecting a pars plicata and an iris root site of the eye.

Example 13 is an assembly, comprising: a source of electromagnetic radiation; a waveguide coupled to the source of electromagnetic radiation for conveying the electromagnetic radiation from a proximal end of the waveguide to a distal end of the waveguide; and a probe having a probe body supporting a portion of the waveguide and a probe tip supporting the distal end of the waveguide, wherein the probe tip is shaped to mate with a surface of an eye at or near a corneal limbus of the eye, and wherein the distal end of the waveguide is oriented within the probe tip to direct the electromagnetic radiation along a treatment path intersecting a Schlemm's canal and trabecular meshwork of the eye.

Example 14 is the assembly of example(s) 13, wherein the waveguide is positioned within the probe tip such that the treatment path further intersects paralimbal tissue of the eye.

Example 15 is the assembly of example(s) 13 or 14, wherein the waveguide is positioned within the probe tip such that the treatment path further intersects corneal tissue of the eye.

Example 16 is the assembly of example(s) 13 or 14, wherein the waveguide is positioned within the probe tip such that the treatment path further intersects scleral tissue of the eye.

Example 17 is the assembly of example(s) 13-16, wherein the waveguide is an optical waveguide and the source of electromagnetic radiation is a light source.

Example 18 is the assembly of example(s) 17, wherein the source of electromagnetic radiation is a laser.

Example 19 is the assembly of example(s) 13-18, wherein the probe tip includes a distal end shaped to mate with a curvature of the eye.

Example 20 is the assembly of example(s) 19, wherein the distal end of the probe tip includes a corneal portion having a curvature that mates with a curvature of a cornea of the eye.

Example 21 is the assembly of example(s) 19 or 20, wherein the distal end of the probe tip includes a scleral portion having a curvature that mates with a curvature of a sclera of the eye.

Example 22 is the assembly of example(s) 13-21, wherein the probe further comprises one or more actuators coupled to the waveguide for adjusting the orientation of the treatment path with respect to the probe tip.

Example 23 is a scleral limbal probe, comprising: a probe tip shaped to mate with a surface of an eye at or near a scleral limbal area of the eye, the eye having a pars plicata and an iris root site; and a waveguide positioned within the probe tip to convey electromagnetic radiation from a source of electromagnetic radiation into the eye, wherein the waveguide is oriented to direct the electromagnetic radiation along a treatment path intersecting the pars plicata and the iris root site.

Example 24 is the probe of example(s) 23, wherein the waveguide is an optical waveguide and the source of electromagnetic radiation is a light source.

Example 25 is the probe of example(s) 24, wherein the source of electromagnetic radiation is a laser.

Example 26 is the probe of example(s) 23-25, wherein the probe tip includes a distal end shaped to mate with a curvature of the eye.

Example 27 is the probe of example(s) 26, wherein the distal end of the probe tip includes a corneal portion having a curvature that mates with a curvature of a cornea of the eye.

Example 28 is the probe of example(s) 26 or 27, wherein the distal end of the probe tip includes a scleral portion having a curvature that mates with a curvature of a sclera of the eye.

Example 29 is the probe of example(s) 23-28, further comprising one or more actuators coupled to the waveguide for adjusting the orientation of the treatment path with respect to the probe tip.

Example 30 is the probe of example(s) 23-29, wherein the source of electromagnetic radiation is housed within a probe body coupled to the probe tip.

1. A paralimbal probe, comprising:
a probe tip shaped to mate with a surface of an eye at or near a corneal limbus of the eye, the eye having a Schlemm's canal and trabecular meshwork; and
a waveguide positioned within the probe tip to convey electromagnetic radiation from a source of electromagnetic radiation into the eye,
wherein the waveguide is oriented to direct the electromagnetic radiation along a treatment path intersecting the Schlemm's canal and the trabecular meshwork.

2. The probe of claim 1, wherein the waveguide is further positioned within the probe tip such that the treatment path further intersects paralimbal tissue of the eye.

3. The probe of claim 1, wherein the waveguide is further positioned within the probe tip such that the treatment path further intersects corneal tissue of the eye or scleral tissue of the eye.

4. (canceled)

5. The probe of claim 1, wherein the waveguide is an optical waveguide and the source of electromagnetic radiation is a light source or a laser.

6. (canceled)

7. The probe of claim 1, wherein the probe tip includes a distal end shaped to mate with a curvature of the eye, and wherein the distal end of the probe tip includes either:
a corneal portion having a curvature that mates with a curvature of a cornea of the eye; or
a scleral portion having a curvature that mates with a curvature of a sclera of the eye.

8.-9. (canceled)

10. The probe of claim 1, further comprising one or more actuators coupled to the waveguide for adjusting the orientation of the treatment path with respect to the probe tip.

11. The probe of claim 1, wherein the source of electromagnetic radiation is housed within a probe body coupled to the probe tip.

12. The probe of claim 1, wherein the probe tip is shaped to mate with a second surface of the eye located anteriorly form the surface of the eye; and wherein the waveguide is oriented within the probe tip to direct additional electromagnetic radiation along an additional treatment path intersecting a pars plicata and an iris root site of the eye.

13. An assembly, comprising:
a source of electromagnetic radiation;
a waveguide coupled to the source of electromagnetic radiation for conveying the electromagnetic radiation from a proximal end of the waveguide to a distal end of the waveguide; and
a probe having a probe body supporting a portion of the waveguide and a probe tip supporting the distal end of the waveguide, wherein the probe tip is shaped to mate with a surface of an eye at or near a corneal limbus of the eye, and wherein the distal end of the waveguide is oriented within the probe tip to direct the electromagnetic radiation along a treatment path intersecting a Schlemm's canal and trabecular meshwork of the eye.

14. The assembly of claim 13, wherein the waveguide is positioned within the probe tip such that the treatment path further intersects paralimbal tissue of the eye.

15. The assembly of claim 13, wherein the waveguide is positioned within the probe tip such that the treatment path further intersects corneal tissue of the eye.

16. The assembly of claim 13, wherein the waveguide is positioned within the probe tip such that the treatment path further intersects scleral tissue of the eye.

17. The assembly of claim 13, wherein the waveguide is an optical waveguide and the source of electromagnetic radiation is a light source or a laser.

18. (canceled)

19. The assembly of claim 13, wherein the probe tip includes a distal end shaped to mate with a curvature of the eye, wherein the distal end of the probe tip includes either:
a corneal portion having a curvature that mates with a curvature of a cornea of the eye; or
a scleral portion having a curvature that mates with a curvature of a sclera of the eye.

20.-21. (canceled)

22. The assembly of claim 13, wherein the probe further comprises one or more actuators coupled to the waveguide for adjusting the orientation of the treatment path with respect to the probe tip.

23. A scleral limbal probe, comprising:
a probe tip shaped to mate with a surface of an eye at or near a scleral limbal area of the eye, the eye having a pars plicata and an iris root site; and
a waveguide positioned within the probe tip to convey electromagnetic radiation from a source of electromagnetic radiation into the eye, wherein the waveguide is oriented to direct the electromagnetic radiation along a treatment path intersecting the pars plicata and the iris root site.

24. The probe of claim 23, wherein the waveguide is an optical waveguide and the source of electromagnetic radiation is a light source or a laser.

25. (canceled)

26. The probe of claim 23, wherein the probe tip includes a distal end shaped to mate with a curvature of the eye, and wherein the distal end of the probe tip includes either:
a comeal portion having a curvature that mates with a curvature of a cornea of the eye; or
a scleral portion having a curvature that mates with a curvature of a sclera of the eye.

27. (canceled)

28. (canceled)

29. The probe of claim 23, further comprising one or more actuators coupled to the waveguide for adjusting the orientation of the treatment path with respect to the probe tip.

30. (canceled)
